(19) United States
(12) Patent Application Publication
(10) Pub. No.: US 2024/0335415 A1
(43) Pub. Date: Oct. 10, 2024

(54) COMPOSITIONS COMPRISING CONSTITUENTS, DERIVATIVES OR EXTRACTS OF CANNABIS

(71) Applicant: Nicoventures Trading Limited, London (GB)

(72) Inventors: Steven ALDERMAN, Winston-Salem, NC (US); Karen TALUSKIE, Winston-Salem, NC (US); Kathryn Lynn WILBERDING, Winston-Salem, NC (US); Jenni HAWKE, London (GB); Ashley DAVIES, London (GB); Thomas H. POOLE, Winston-Salem, NC (US); Michael DANIEL, Winston-Salem, NC (US); Kai TANG, Winston-Salem, NC (US); Keyi XU, Winston-Salem, NC (US); Karina MCQUILLAN, London (GB); John CARAWAY, Winston-Salem, NC (US)

(21) Appl. No.: 18/579,786

(22) PCT Filed: Jul. 21, 2022
(86) PCT No.: PCT/GB2022/051900

§ 371 Date: Jan. 16, 2024

Related U.S. Application Data
(60) Provisional application No. 63/224,570, filed on Jul. 22, 2021.

(51) Int. Cl. A61K 31/352; A61K 47/40

(52) U.S. Cl. CPC A61K 31/352; A61K 47/40

(57) ABSTRACT

The present invention relates to compositions comprising constituents, derivatives or extracts of cannabis. The invention also relates to uses of such compositions and methods of manufacturing them.

### TECHNICAL FIELD

The present invention relates to compositions comprising constituents, derivatives or extracts of cannabis. The invention also relates to uses of such compositions and methods of manufacturing them.

### BACKGROUND

Oral products comprising cannabinoids are known and seek to deliver the cannabinoids to the user.

### SUMMARY

According to a first aspect of the present invention, there is provided a composition comprising one or more constituent, derivative or extract of cannabis, a lipid or a solvent in which the one or more constituent, derivative or extract of cannabis is at least partially soluble, and an additive which slows or inhibits crystallisation of the one or more constituent, derivative or extract of cannabis.

In some embodiments, the composition comprises a liquid in which the one or more constituent, derivative or extract of cannabis is at least partially dissolved.

In some embodiments, the lipid is one or more selected from the group consisting of: oils, long or short chain fatty acids, which may be saturated or unsaturated, and long or short chain triglycerides.

In some embodiments, the solvent comprises an amphiphilic component such as an alcohol.

In some embodiments, the one or more constituent, derivative or extract of cannabis is encapsulated.

In some embodiments, the encapsulation is by a molecular encapsulant, such as a cyclodextrin.

In some embodiments, the encapsulation is by a micelle comprising a surfactant.

In some embodiments, the surfactant is selected from the group consisting of long chain triglycerides (such as C16-C18 triglycerides), linoleic acid, glyceryl monooleate; and sodium lauryl sulfate (sodium dodecyl sulfate, SLS, or SDS), docusate sodium, lecithin, polyoxyethylene sorbitan fatty acid esters (Polysorbate, Tween®), polyoxyethylene 15 hydroxy stearate (Macrogol 15 hydroxy stearate, Solutol HS₁₅®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®) and sugar esters.

In some embodiments, the composition comprises surfactant in an amount of from about 0.5 to about 1.5% by weight, based on the total weight of the composition.

In some embodiments, wherein the additive is selected from the group consisting of polyvinylpyrrolidone (PVP), hydroxypropyl cellulose (HPC), methyl cellulose (MC), hydroxypropyl methyl cellulose (HPMC), poloxamer (F68), polyvidon, Hydroxypropyl methylcellulose acetate succinate (HPMC-AS), or a combination thereof.

In some embodiments, the one or more constituent, derivative or extract of cannabis is selected from the group consisting of: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

In some embodiments, the constituent, derivative or extract of cannabis is present in an amount of from about 0.1 to about 30% by weight, based on the total weight of the composition.

In some embodiments, the composition has enhanced bioavailability properties in the gastric system.

In some embodiments, the composition comprises one of more components enhancing enterocyte intestinal absorption of the one or more constituent, derivative or extract of cannabis.

In some embodiments, the composition comprises one of more components increasing intestinal lymphatic transport of the one or more constituent, derivative or extract of cannabis.

In some embodiments, the composition comprises a terpene, a grapefruit extract, piperine or a black pepper extract.

In some embodiments, the composition comprises a pH modifier.

In some embodiments, the composition comprises a flavour or sensate.

In some embodiments, the composition comprises a further active agent.

In some embodiments, the composition is in the form of a solid unit dosage form, a powder or granules.

In some embodiments, the composition is water dispersible.

In some embodiments, the composition is in the form of particles having a volume mean diameter of from about 50 to about 500 μm.

In some embodiments, the composition is a solution or colloidal dispersion.

According to a second aspect of the present invention, there is provided a method for preparing a composition, the method comprising combining: one or more constituent, derivative or extract of cannabis; a lipid or a solvent in which the one or more constituent, derivative or extract of cannabis is at least partially soluble; and an additive which slows or inhibits crystallisation of the one or more constituent, derivative or extract of cannabis.

In some embodiments, the combination of the one or more constituent, derivative or extract of cannabis, the lipid or solvent in which the one or more constituent, derivative or extract of cannabis is at least partially soluble, and the additive which slows or inhibits crystallisation of the one or more constituent, derivative or extract of cannabis is dried to form a water soluble or water dispersible solid composition.

In some embodiments, the combination is spray dried to form the composition.

According to a third aspect of the present invention, there is provided a beverage for providing gastric delivery of one or more constituent, derivative or extract of cannabis, comprising the composition.

According to a fourth aspect of the present invention, there is provided a use of the composition to form a beverage for providing gastric delivery of one or more constituent, derivative or extract of cannabis by introducing the composition into a liquid.

### DETAILED DESCRIPTION

The present invention seeks to provide compositions comprising one or more constituent, derivative or extract of cannabis for enteral delivery.

The compositions are intended for human use. They may also be configured for oral use and deliver the constituent, derivative or extract of cannabis, as well as optionally other substances such as flavours and/or active ingredients during use.

In some embodiments, the constituent, derivative or extract of cannabis is a cannabinoid. Cannabinoids are lipid-soluble, hydrophobic molecules that are insoluble or poorly soluble in water.

The provision of solubilised constituents, derivatives or extracts of cannabis in an aqueous system, for example in a beverage, is technically challenging due to the lipophilic nature of the compounds in the cannabinoid family. This means that constituents, derivatives or extracts of cannabis are poorly soluble in aqueous environments. They are, however, soluble in a lipid component or in some solvents, such as ethanol.

Another common issue associated with the gastric delivery of compositions comprising constituents, derivatives or extracts of cannabis is the bioavailability of the active substance. Gastric, or more accurately, enteral delivery generally enjoys the benefits of high intestinal surface area and rich mucosal vasculature to provide good absorption and bioavailability. However, for constituents, derivatives or extracts of cannabis that are poorly soluble in the aqueous environment of the small and large intestines, it is challenging to present these compounds in a form that will allow them to be absorbed across the mucosa. Further, once absorbed, the majority of constituent, derivative or extract of cannabis will be transported via the portal vein to the liver where it will undergo first-pass metabolism and so will not provide the desired physiological effects.

Another problem that this invention seeks to overcome is the bitter taste associated with constituents, derivatives or extracts of cannabis. The inclusion of constituent, derivative or extract of cannabis into compositions such as beverages or other edible forms is commonly overlooked due to their bitter taste which consumers often find off-putting.

The present invention relates to the provision of a composition comprising one or more constituent, derivative or extract of cannabis, a lipid or a solvent in which the one or more constituent, derivative or extract of cannabis is at least partially soluble, and an additive which slows or inhibits crystallisation of the one or more constituent, derivative or extract of cannabis in an aqueous environment.

In some embodiments the one or more constituent, derivative or extract of cannabis is at least partially dissolved or solubilised in a lipid or solvent in the composition. In some embodiments the one or more constituent, derivative or extract of cannabis is at least partially dissolved or solubilised in a lipid or non-polar solvent when the composition is exposed to an aqueous environment, such as in an aqueous liquid, or in the oral cavity or the gastrointestinal tract.

In some embodiments, the compositions and their use are intended to provide enteral delivery of the one or more constituent, derivative or extract of cannabis.

In some embodiments, the components of the composition enhance the bioavailability of the one or more constituent, derivative or extract of cannabis, by one of more of the following mechanisms: (i) increasing solubility of the one or more constituent, derivative or extract of cannabis in the aqueous environment of the gastrointestinal tract; (ii) reducing the tendency of the one or more constituent, derivative or extract of cannabis to recrystallize in an aqueous environment; (iii) enhancing enterocyte intestinal absorption of the one or more constituent, derivative or extract of cannabis; and (iv) increasing intestinal lymphatic transport.

In some embodiments, the compositions is incorporated into an oral product, such as a beverage which may be provided by introducing the composition into an aqueous liquid. The composition may be delivered to the gastric system via an oral route. The composition may optionally comprise other substances such as flavours and/or active ingredients.

### Enteral Delivery

The compositions provided herein are intended for oral administration, and in some embodiments, the intention is for the majority of the constituent, derivative or extract of cannabis provided in the composition to have their effect by enteral absorption through the intestines. A small proportion of the one or more constituent, derivative or extract of cannabis may be absorbed through the mucosa in the mouth (e.g., via buccal or sublingual delivery), but most will be swallowed and should be taken up by enterocyte intestinal absorption (i.e., via enteral delivery).

Enteral delivery provides a delayed effect of the constituents, derivatives or extracts of cannabis. In some embodiments, the delayed effect of the one or more constituent, derivative or extract of cannabis may be combined with a flavour, sensate or other active ingredient that has a more rapid effect, as described herein. In such embodiments, the other ingredient may deliver its effect instantly or very shortly after administration of the composition, whilst the constituent, derivative or extract of cannabis may then deliver its effect afterwards. This advantageously provides the user with two consecutive effects.

The time from administration of the composition to the effect of the constituent, derivative or extract of cannabis may be at least about 5 minutes, at least about 10 minutes, at least about 20 minutes, at least about 30 minutes.

In some embodiments, the composition is at least partially dissolved in the saliva of the mouth. Said saliva may be at least partially swallowed, providing gastric delivery of the composition.

### The Aqueous Environment

As used herein, the aqueous environment may refer to a liquid that the composition is added to during use or consumption. In some embodiments, this may be a liquid that is included in the composition or to a liquid that the composition is added to, for example to create a beverage. Further, the aqueous environment may also refer to the aqueous conditions in the gastrointestinal tract, including the oral cavity.

### Aqueous Compositions

In some embodiments, the composition comprises an aqueous component. Such embodiments, include, for example, compositions that are formulated as a liquid or a suspension to be consumed as a beverage or the like.

The aqueous component may be beneficial to the invention by enabling the dilution of the other components of the composition. This may be advantageous for the consumer, as the composition is readily swallowed and the flavour of the one or more constituent, derivative or extract of cannabis may be less localised and can be diluted and/or masked. This may reduce any bitter or negative flavours associated with the one or more constituent, derivative or extract of cannabis, and provide the consumer with a suitable effect.

### Soluble or Dispersible Compositions

In some embodiments, the composition is in solid form. For example, the composition may be in the form of a tablet, granules or a powder. The composition may also be a capsule with a solid component and a liquid component.

Such a composition may be dissolved or dispersed in a liquid to form a liquid formulation for consumption. For example, the composition may be added to a liquid which may or may not comprise an aqueous component, to form a beverage. For such an application, the composition should be readily soluble or dispersible in the liquid, so that it quickly forms the liquid formulation upon addition to or mixing with the liquid. In some embodiments, the liquid to which the composition is added may comprise a lipid or solvent. The beverage provided or formed may be a two-phase system. In some embodiments, the two-phase system may be a colloid, such as an emulsion.

In other embodiments, the solid composition may be intended for direct oral administration. This means that the composition is ingested in solid form. In such embodiments, the solid composition may be formulated or administered to be at least dissolved or dispersed in the aqueous environment of the oral cavity. Alternatively, the composition may be formulated or administered to be dissolved or dispersed in the aqueous environment of the stomach and/or intestines.

In some embodiments, the composition dissolves rapidly in an aqueous environment, providing the advantage that a consumable product such as a beverage may be prepared quickly by the user when a solid composition comprising the constituent, derivative or extract of cannabis is added to a liquid before being administered, or so that a solid composition rapidly dissolves in the gastrointestinal tract after being administered in its solid form, as described herein.

In some embodiments, the composition is soluble or dispersible in the aqueous environment which it is at a temperature of at least about 20° C., about 25° C., about 30° C., about 40° C., about 50° C., about 60° C., about 70° C., about 80° C., or about 85° C. This means that the consumer can add the composition in the form of a solid dosage (described herein) to a warm beverage, which will provide an additional sensation when the beverage is drunk.

### Constituent, Derivative or Extract of Cannabis

As used herein, any compound or mixture of compounds which may be obtained from cannabis may be a constituent, derivative or extract thereof, including synthetic versions of such compound(s) or such compound(s) derived from other natural sources.

In some embodiments the constituent, derivative or extract of cannabis comprises, or is, one or more compounds selected from: cannabinoids (such as phytocannabinoids that may optionally be THC and/or CBD); terpenes (such as triterpenes); alkaloids; and flavonoids.

In some embodiments the constituent, derivative or extract of cannabis comprises one or more compounds selected from: cannabinoids (such as phytocannabinoids) and terpenes (such as triterpenes).

In some embodiments the constituent, derivative or extract of cannabis comprises one or more cannabinoids, such as phytocannabinoids.

Cannabinoids are a class of natural or synthetic chemical compounds which act on cannabinoid receptors (i.e., CB1 and CB2) in cells that repress neurotransmitter release in the brain. Cannabinoids may be naturally occurring (phytocannabinoids) from plants such as cannabis, from animals (endocannabinoids), or artificially manufactured (synthetic cannabinoids). Cannabis species express at least 85 different phytocannabinoids, and are divided into subclasses, including cannabigerols, cannabichromenes, cannabidiols, tetrahydrocannabinols, cannabinols and cannabinodiols, and other cannabinoids. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

In some embodiments, the cannabinoids are phytocannabinoids.

In some embodiments, the terpenes are triterpenes.

In particular embodiments, the constituent, derivative or extract of cannabis comprises, or is, tetrahydrocannabinol (THC) and/or cannabidiol (CBD).

In some embodiments, the constituent, derivative or extract of cannabis comprises, consists of or essentially consists of THC.

In particular embodiments, the constituent, derivative or extract of cannabis comprises, consists of or essentially consists of CBD.

In some embodiments, the cannabinoid is selected from tetrahydrocannabinol (THC), the primary psychoactive compound in cannabis, and cannabidiol (CBD) another major constituent of the plant, but which is devoid of or provides different psychoactivity. All of the above compounds can be used in the form of an isolate from plant material or synthetically derived. The selection of constituent, derivative or extract of cannabis may depend on the desired effect on the user, as well as other factors such as taste, solubility, bioavailability, and current regulations on the substances.

In some embodiments, the constituent, derivative or extract of cannabis is present in an amount of from about 0.1 to about 30% by weight, based on the total weight of the composition.

The particular percentages of constituent, derivative or extract of cannabis present will vary depending upon the desired characteristics of the particular product. In some embodiments of the invention, the composition comprises from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% of at least one constituent, derivative or extract of cannabis by weight, based on the total weight of the composition.

In some embodiments of the invention, the composition comprises at most about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% of at least one constituent, derivative or extract of cannabis by weight.

In some embodiments of the invention, the composition comprises about 2 to about 10 mg, about 10 to about 20 mg, about 20 to about 30 mg, about 30 to about 40 mg, about 40 to about 50 mg, or about 50 to about 60 mg of at least one constituent, derivative or extract of cannabis.

The composition may contain a high concentration of the constituent, derivative or extract of cannabis. This means a high delivery levels of the active ingredient can be achieved. The high concentration of the constituent, derivative or extract of cannabis also improves the ability to deliver a sufficient quantity of active to the user without increasing the amount of the composition or the total volume of the aqueous environment. In addition, a smaller amount of the composition is required to be consumer for the user to achieve the desired effect. Typically, a consumer may consume at most 60 mg/day of a constituent, derivative or extract of cannabis.

### Increasing Solubility of the Constituent, Derivative or Extract of Cannabis

### Lipids and Solvents

When present in the composition, a solvent may be polar or non-polar. In some embodiments, the composition comprises a solvent with amphiphilic properties. Examples of such solvents include ethanol.

When present in the composition, a lipid may be solid or liquid at room temperature. Examples of lipids that are solid at room temperature include saturated and unsaturated fats.

In some embodiments, the lipid may be an oil. In some embodiments, the lipid comprises an oil and, in particular, a food grade oil including fractionated oils. In some embodiments, the lipid comprises a combination of oils.

Such oils include, but are not limited to, vegetable oils (e.g., acai oil, almond oil, amaranth oil, apricot oil, apple seed oil, argan oil, avocado oil, babassu oil, beech nut oil, ben oil, bitter gourd oil, black seed oil, blackcurrant seed oil, borage seed oil, borneo tallow nut oil, bottle gourd oil, brazil nut oil, buffalo gourd oil, butternut squash seed oil, cape chestnut oil, canola oil, carob cashew oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, marula oil, meadowfoam seed oil, mongongo nut oil, mustard oil, niger seed oil, nutmeg butter, okra seed oil, olive oil, orange oil, palm oil, papaya seed oil, peanut oil, pecan oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof), animal oils (e.g., cattle fat, buffalo fat, sheep fat, goat fat, pig fat, lard, camel fat, tallow, liquid margarine, fish oil, fish liver oil, whale oil, seal oil, and combinations thereof), and mineral oils.

In some embodiments, one or more oil is selected from the group consisting of mineral oil, vegetable oil, canola oil, olive oil, fish oils, omega 3 oil or any other edible oils. The advantages of such oils is that the constituent, derivative or extract of cannabis is readily soluble, making manufacturing more easy and efficient. The oil itself may also have advantages, such as health benefits. In such embodiments, the oil may be portioned in a bottle, sachet, capsule or any other portioning packaging.

In some embodiments, the composition is an edible item, such as chocolate, a milk-based beverage, an alcoholic beverage, a melt or other items comprising a lipid or solvent based component.

In some embodiments, the composition comprises a lipid based system, or a lipid-dispersion system. Examples of such lipid or lipid-dispersion systems may comprise long or short chain fatty acids, which may be saturated or unsaturated. Such lipids may be long or short chain triglycerides. In particular, long chain triglycerides enjoy the advantage of high constituent, derivative or extract of cannabis solubility.

In some embodiments, the composition comprises a bi-phasic system, comprising an aqueous and a non-aqueous component. In some embodiments, such systems also comprise an amphiphilic component. Such amphiphilic components may include alcohols such as ethanol or emulsifiers, as described herein.

### Encapsulation

In order to further increase the solubility of the constituent, derivative or extract of cannabis, they may be encapsulated. This encapsulation may be within the composition and/or it may occur when the constituent, derivative or extract of cannabis is exposed to or released into the aqueous environment.

It is may also be desirable to encapsulate constituent, derivative or extract of cannabis to mask its bitter taste. An additional advantage of encapsulation, if that it increase the bioavailability of the constituent, derivative or extract of cannabis, and directs the constituent, derivative or extract of cannabis to intestinal lymphatic system delivery. Various exemplary embodiments of encapsulation methods may be used and are described herein.

In some embodiments, the constituent, derivative or extract of cannabis may be encapsulated by the lipid present in the composition.

In some embodiments, the constituent, derivative or extract of cannabis is encapsulated in a micelle. A micelle is an aggregation, assembly or “shell” of surfactant molecules dispersed in a liquid, forming a colloidal suspension. The surfactant may be selected from the group consisting of long chain triglycerides, such as C16-C18 triglycerides, and linoleic acid for example. In some embodiments the surfactant is advantageously selected for its safety (which may be food grade), perceived “natural” association, taste, and consumer perception.

Examples of suitable surfactants include: long chain triglycerides, such as C16-C18 triglycerides, linoleic acid, glyceryl monooleate; and sodium lauryl sulfate (sodium dodecyl sulfate, SLS, or SDS), docusate sodium, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®), sugar esters and lecithins.

The encapsulation of the constituent, derivative or extract of cannabis in micelles enjoys the advantage that this aids solubility in aqueous environments. This is because the micelle may have a non-polar core in which the constituent, derivative or extract of cannabis is soluble, and a polar exterior, making it more soluble in aqueous environments. In addition, the micelle can provide stable conditions for the constituent, derivative or extract of cannabis. This can prevent the constituent, derivative or extract of cannabis deteriorating. For example, this may provide suitable environment to inhibit the conversion of CBD to THC. This may be particularly suitable in jurisdictions with regulations around specific constituents, derivatives or extracts of cannabis.

The micelle may also provide advantageous flavour improvements. Micelles are effective for mitigating both bitterness and throat burning as they provide a layer around the constituent, derivative or extract of cannabis, whilst still maintaining its bioavailability in the gastric system. In addition, micellar encapsulation also allows higher concentrations of the constituent, derivative or extract of cannabis in the aqueous environment. This is due to the increased solubility as well as the taste-masking qualities.

Finally, micellar encapsulation is known to maintain the homogenous nature of the solution once the composition is solubilised in the aqueous environment. This means that the solution is less likely or slower to separate oil and aqueous phases.

In some embodiments, said micelles may be formed via autoencapsulation. The benefit of this method is the ability to solubilise large volumes.

In some embodiments, cyclodextrin is used as an encapsulating agent. Cyclodextrins have a lipophilic central cavity and an outer hydrophilic shell. Thus, the cyclodextrin is able to form water-soluble inclusion complexes with the poorly soluble constituent, derivative or extract of cannabis. Formulation with cyclodextrins may also improve the physical and chemical stability of the constituent, derivative or extract of cannabis.

The cyclodextrin may be selected from the group of α-to β-and γ-cyclodextrin for example. In some embodiments, the cyclodextrin is essentially γ-cyclodextrin. γ-cyclodextrin provides the benefits that is a wider, nonplanar and more flexible structure. This means that γ-cyclodextrin is more water soluble. In addition, γ-cyclodextrin binds most favourably to the constituent, derivative or extract of cannabis as it fits in the cavity. In some embodiments, the cyclodextrin is methylated, hydroxyalkylated, acetylated, and sulfobutylated.

There are multiple techniques to encapsulate the constituent, derivative or extract of cannabis inside a chosen cyclodextrin, of which freeze drying and lyophilisation may be particularly efficient.

### Micro-Emulsion

In some embodiments, the constituent, derivative or extract of cannabis is provided in a micro-emulsion. The micro-emulsion may comprise an aqueous solution, the constituent, derivative or extract of cannabis and an amphiphilic material or emulsifier. Micro-emulsions provide the benefit the formation of a micro-emulsion is spontaneous (driven by surface tension, entropy gain, and thermodynamic gain), and so is advantageous for manufacturing the composition. In addition, micro-emulsions have improved long-term stability and are slow to separate, as this is thermodynamically unfavourable.

In certain embodiments, an emulsifier may be added to the composition. In some embodiments, the emulsifier is lecithin. For example, lecithin (e.g., soy lecithin or sunflower lecithin) may be added to the composition to provide smoother textural properties to the composition and to improve flowability and mixing of e.g., a lipid with the remaining components of the composition. Emulsifiers (e.g., lecithin) can be used in an amount of about 0.01 to about 5% by dry weight of the composition, such as from about 0.1 to about 2.5%, or from about 0.5 to about 1.5% based on the total weight of the composition.

### Reducing Recrystallization

The composition may comprise an additive which slows or inhibits crystallisation of the one or more constituent, derivative or extract of cannabis. As discussed herein, constituents, derivatives or extracts of cannabis are associated with poor solubility in aqueous systems due to their lipophilic nature. Even when solubilised, constituents, derivatives or extracts of cannabis are quick to recrystallize when in an aqueous environment, which significantly impairs their bioavailability and thus their efficacy. In order to mitigate this, an additive may be added to prevent or inhibit the constituent, derivative or extract of cannabis crystallising or coming out of solution. This is particularly advantageous when spray-drying is used as the encapsulation method.

As described herein, encapsulation of the constituent, derivative or extract of cannabis may also inhibit, slow or prevent crystallisation. In some embodiments, the constituent, derivative or extract of cannabis may be encapsulated by the additive via molecular or micellar encapsulation for example.

Alternatively, the following additives may be provided in the composition.

Examples of suitable additive which slow or prevent crystallisation include polyvinylpyrrolidone (PVP), hydroxypropyl cellulose (HPC), methyl cellulose (MC), hydroxypropyl methyl cellulose (HPMC), poloxamer (F68), polyvidon, Hydroxypropyl methylcellulose acetate succinate (HPMC-AS), or a combination thereof.

In addition or alternatively, the additive to inhibit crystallisation may include one or more surfactants. Examples of suitable surfactants include: long chain triglycerides, such as C16-C18 triglycerides, linoleic acid, glyceryl monooleate; and sodium lauryl sulfate (sodium dodecyl sulfate, SLS, or SDS), docusate sodium, lecithin, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®) and sugar esters. Such surfactants may be in the form of an encapsulant, which encapsulates the constituent, derivative or extract of cannabis.

In some embodiments, the surfactant is present in an amount of from about 0.5 to about 10% by weight, based on the total weight of the composition.

### Enhancing Intestinal Absorption

Orally administered active substances typically show low bioavailability due to their degradation by enzymes in the gastrointestinal tract, and due to the difficulty of absorbing them in the small intestine.

Additives may be included in the compositions to promote enteral absorption.

For example, medium chain fatty acid salts may enhance enteral delivery of the constituent, derivative or extract of cannabis by increasing paracellular permeability of the intestinal epithelium.

Liposomes are vesicles formed from lipid bilayers made up of one or more types or lipid. The bilayer provides a hydrophobic environment whilst there can be an aqueous phase inside the vesicle. Hydrophilic molecules can be loaded into the interior of liposomes, whilst hydrophobic or lipophilic molecules, such as constituents, derivatives or extracts of cannabis, are incorporated into the lipid bilayer of the liposome.

Liposomes have the ability to encapsulate and protect active substances and to increase their absorption into enterocytes. Liposomes can protect labile active substances from denaturation by the harsh conditions in the gastrointestinal tract. The lipids of liposomes can also be utilized to stimulate the production of chylomicrons in enterocytes, thus enhancing drug transport into the lymphatic system. Furthermore, enterocyte uptake of liposomes can be controlled with their size; smaller showed higher uptake.

Suitable liposome-forming lipids include, for example, phospholipids such as phosphatidylcholine. Other lipids may also be used.

### Increasing Intestinal Lymphatic Transport

Following oral administration and enteral absorption, the constituents, derivatives or extracts of cannabis will either enter the portal vein or intestinal lymphatic system. The main factors that control the route that they take are molecular mass and solubility. It has been found that constituents, derivatives or extracts of cannabis are preferentially transported via the portal vein. There, they immediately accumulate in the liver and are then metabolized by enzymes, which lowers their concentration in the bloodstream.

The alternative route for delivering active substances to the systemic circulation is the intestinal lymphatic pathway. The intestinal lymphatic pathway can bypass first-pass metabolism in the liver, thus increasing bioavailability. It is therefore desirable to increase intestinal lymphatic transport of the constituents, derivatives or extracts of cannabis.

As mentioned above, liposomes may not only enhance enteral delivery but also enhance transport to the lymphatic system.

Co-administration of lipids generally may also enhance transport of the constituents, derivatives or extracts of cannabis to the lymphatic system. Therefore, in some embodiments, the composition comprises a lipid component. The lipids may include, for example, phospholipids, long-chain triglycerides and fatty acids such as oleic acid.

In some embodiments, the composition comprises an additive that acts as a metabolism-directing agent, enhancing transport to the lymphatic system. Suitable metabolism directing agents include terpenes, a grapefruit extract, piperine (which is typically extracted from black pepper), or combinations thereof. The metabolism-directing agent advantageously direct the constituent, derivative or extract of cannabis to the gastric system. This may alter the time for the constituent, derivative or extract of cannabis or active ingredient to take effect. In addition, the particle size also has an effect of the metabolism of the constituent, derivative or extract of cannabis, and this may be selected advantageously to have a synergistic effect with the metabolism directing agent.

Some terpenes provide an entourage effect when used in combination with constituents, derivatives or extracts of cannabis or cannabimimetics.

In some embodiments, the metabolism-directing agent is a terpene. Terpenes are understood to have the general formula of (C₅H₈)ₙ and include monoterpenes, sesquiterpenes, and diterpenes. Terpenes can be acyclic, monocyclic or bicyclic in structure. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

An advantage of the inclusion of terpenes is that many terpenes are associated with biological effects, such as calming effects. This can provide the user with an additional effect, on top of that which is provided with the constituent, derivative or extract of cannabis. The rate of metabolism of the terpene may also be different to the constituent, derivative or extract of cannabis, so as to provide the user with an effect at different times.

### Additional Actives

In some embodiments, the composition comprises one or more active substance in addition to the one or more constituent, derivative or extract of cannabis.

The further active substance as used herein may be a physiologically active material, which is a material intended to achieve or enhance a physiological response. The active substance may for example be selected from nutraceuticals, nootropics, and psychoactives. The active substance may be naturally occurring or synthetically obtained.

In some embodiments, the one or more additional active ingredients, may include, for example: botanical ingredients, stimulants, amino acids, nicotine components, pharmaceutical ingredients, nutraceutical ingredients, medicinal ingredients, terpenes, and combinations thereof.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 25 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided below.

### Terpenes

Active ingredients suitable for use in the present disclosure can also be classified as terpenes, many of which are associated with biological effects, such as calming effects.

Terpenes are understood to have the general formula of (C₅H₈)ₙ and include monoterpenes, sesquiterpenes, and diterpenes. Terpenes can be acyclic, monocyclic or bicyclic in structure.

Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods”. Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. One example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition;

### Amino Acids

In some embodiments, the active ingredient comprises an amino acid. As used herein, the term “amino acid” refers to an organic compound that contains amine (—NH2) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification).

Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins and Minerals

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B₃ (niacin), vitamin B₅ (pantothenic acid), vitamin B6 (pyridoxine), vitamin B₇ (biotin), vitamin B₉ (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5%, about 0.6%, about 0.7%, about 0.8%, about 0.9%, about 1%, about 2%, about 3%, about 4%, about 5%, or about 6% by weight, based on the total weight of the composition.

In some embodiments, the active ingredient comprises a mineral or combination of minerals. As used herein, the term “mineral” refers to a chemical compound with a defined chemical composition and a specific crystal structure that occurs naturally in pure form. In some embodiments, the active ingredient comprises a magnesium-based mineral compounds, e.g., such as magnesium gluconate, magnesium citrate, and the like. When present, a mineral or combination of minerals (e.g., magnesium gluconate, magnesium citrate, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage.

Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytosterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Nicotine Component

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12:43-54 (1983), which are incorporated herein by reference.

Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrylic acid, such as Amberlite IRP6₄, Purolite C11₅HMR, or Doshion P₅₅1. See, for example, U.S. Pat. No. 3,901,248 to Lichtneckert et al., which is incorporated herein by reference. Another example is a nicotine polyacrylic carbomer complex, such as with Carbopol ₉₇₄P. In some embodiments, nicotine may be present in the form of a nicotine polyacrylic complex.

Typically, the nicotine component (calculated as the free base) when present, is in a concentration of at least about 0.001% by weight of the composition, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl) propanoic acid), phosphatidylserine, myoinositol, docosahexaenoic acid (DHA, Omega-3), arachidonic acid (AA, Omega-6), S-adenosylmethionine (SAM), beta-hydroxy-betamethylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin.

In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Sensory Additives

### Flavouring Agents

In some embodiments, the composition comprises a flavouring agent. A flavouring agent may aid masking the bitter taste associated with constituents, derivatives or extracts of cannabis and/or improving its flavour. A flavour may also advantageously complement the flavours of the constituent, derivative or extract of cannabis.

As used herein, a “flavouring agent,” “flavour” or “flavourant” is any flavourful or aromatic substance capable of altering the sensory characteristics associated with the oral product. Examples of sensory characteristics that can be modified by the flavouring agent include taste, mouthfeel, moistness, coolness/heat, and/or fragrance/aroma. Flavouring agents may be natural or synthetic, and the character of the flavours imparted thereby may be described, without limitation, as fresh, sweet, herbal, confectionary, floral, fruity, or spicy. Specific types of flavours include, but are not limited to, vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, trigeminal sensates, terpenes, and any combinations thereof. See also, Leffingwell et al., Tobacco Flavoring for Smoking Products, R. J. Reynolds Tobacco Company (1972), which is incorporated herein by reference. Flavouring agents also may include components that are considered moistening, cooling or smoothening agents, such as eucalyptus. These flavours may be provided neat (i.e., alone) or in a composite, and may be employed as concentrates or flavour packages (e.g., spearmint and menthol, orange and cinnamon, lime, pineapple, and the like).

Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavouring agent may be provided in a spray-dried form or a liquid form.

In some embodiments, the composition may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-_{3.}

A particular advantage of the composition comprising a sensate is that this may provide an additional experience whilst drinking a beverage comprising the constituent, derivative or extract of cannabis. For example, a throat burn sensation is associated with the constituent, derivative or extract of cannabis and may be mitigated or enhanced using a sensate to provide the consumer with an advantageous sensation.

The amount of flavouring agent utilized in the composition can vary, but is typically up to about 10% by weight, and certain embodiments are characterized by a flavouring agent content of at least about 0.1% by weight, such as about 0.5 to about 10%, about 1 to about 5%, or about 2 to about 4% weight, based on the total weight of the composition.

### Taste Modifiers

In order to improve the organoleptic properties of a composition as disclosed herein, the composition may include one or more taste modifying agents (“taste modifiers”) which may serve to mask, alter, block, or improve e.g., the flavour of a composition as described herein. Non-limiting examples of such taste modifiers include analgesic or anaesthetic herbs, spices, and flavours which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the composition may comprise a cannabinoid or other component having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., a cannabinoid). Suitable taste modifiers include, but are not limited to, capsaicin, gamma-amino butyric acid (GABA), adenosine monophosphate (AMP), lactisole, or a combination thereof.

When present, a representative amount of taste modifier is about 0.01% by weight or more, about 0.1% by weight or more, or about 1.0% by weight or more, but will typically make up less than about 10% by weight of the total weight of the composition or aqueous environment, (e.g., from about 0.01%, about 0.05%, about 0.1%, or about 0.5%, to about 1%, about 5%, or about 10% by weight of the total weight of the composition).

### Sweeteners

In order to improve the sensory properties of the composition according to the disclosure, one or more sweeteners may be added. The sweeteners can be any sweetener or combination of sweeteners, in natural or artificial form, or as a combination of natural and artificial sweeteners.

Examples of natural sweeteners include fructose, sucrose, glucose, maltose, mannose, galactose, lactose, isomaltulose, stevia, honey, and the like. Examples of artificial sweeteners include sucralose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form.

Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is sucralose, acesulfame K, or a combination thereof.

A sweetener provide the consumer with a pleasant taste during consumption of the composition or formulation of composition in an aqueous environment. This is particularly important in the present invention to mitigate the bitter flavour associated with constituents, derivatives or extracts of cannabis.

When present, a sweetener or combination of sweeteners may make up from about 0.01 to about 20% or more of the of the composition by weight, for example, from about 0.01 to about 0.1, from about 0.1 to about 1%, from about 1 to about 5%, from about 5 to about 10%, or from about 10 to about 20% by weight, based on the total weight of the composition. In some embodiments, a combination of sweeteners is present at a concentration of from about 0.01% to about 0.1% by weight of the composition, such as about 0.01, about 0.02, about 0.03, about 0.04, about 0.05, about 0.06, about 0.07, about 0.08, about 0.09, or about 0.1% by weight of the composition. In some embodiments, a combination of sweeteners is present at a concentration of from about 0.05% to about 0.5% by weight of the composition, such as about 0.1, about 0.2, about 0.3, about 0.4, or about 0.5% by weight of the composition. In some embodiments, a combination of sweeteners is present at a concentration of from about 1% to about 3% by weight of the composition.

### Salts

In some embodiments, the composition comprises a salt (e.g., an alkali metal salt), typically employed in an amount sufficient to provide desired sensory attributes to the composition. Non-limiting examples of suitable salts include sodium chloride, potassium chloride, ammonium chloride, flour salt, sodium acetate, sodium citrate, calcium citrate, and the like. In some embodiments, the salt is sodium chloride, ammonium chloride, or a combination thereof. In some embodiments, the salt is trisodium citrate, calcium citrate, or a combination thereof.

When present, a representative amount of salt is about 0.1% by weight or more, about 0.5% by weight or more, about 1.0% by weight or more, or about 1.5% by weight or more, but will typically make up about 10% or less of the total weight of the composition, or about 7.5% or less, or about 5% or less (e.g., from about 0.1 to about 5% by weight or from about 0.5 to about 1.5%).

### Other Components

In some embodiments, the composition may further comprise additional active ingredients, functional components, binders, flowability enhancers, organic acids, water, additional actives, sweeteners, salts, flavours, buffers, emulsifiers, bulk carriers, colorants, processing aids, and combinations thereof.

The relative amounts of the various components within the composition may vary, and typically are selected so as to provide the desired sensory and performance characteristics to the composition.

### Preservatives

In some embodiments, the composition or aqueous environment may further comprise a preservative or anti-microbial agent. Examples of such preservatives or antimicrobial agents include: benzyl alcohol, cetylpyridine chloride; glycerin; methyl paraben; propylene glycol; propylene paraben; potassium sorbate; sodium benzoate; sorbic acid; sodium propionate or a combination of these. The inclusion of a preservative(s) or an antimicrobial agent(s) provides the advantage that the aqueous environment or beverage may remain potable for longer, as well as be suitable for storage and transport.

When present, one or more preservative is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition or aqueous environment.

### Stabilisers

In some embodiments, the composition may further comprise a stabiliser to stabilize aqueous against aggregation, gelation, creaming, flocculation, coalescence, drainage, coarsening and other destabilization processes. The stabiliser may be selected from at least of the group consisting of hydrocolloids, proteins, amphiphilic polysaccharides such as whey protein isolate and arabic gums, or a combination thereof.

When present, one or more stabiliser is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition or aqueous environment.

### pH Modifier

In certain embodiments, the composition or aqueous environment can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Non-limiting examples of suitable buffers include alkali metals acetates, glycinates, phosphates, glycerophosphates, citrates, carbonates, hydrogen carbonates, borates, or mixtures thereof. In some embodiments, the buffer is sodium bicarbonate.

Where present, the buffering agent is typically present in an amount less than about 5% by weight, based on the weight of the composition, for example, from about 0.1% to about 5%, such as, e.g., from about 0.1% to about 1%, or from about 0.1% to about 0.5% by weight, based on the total weight of the composition or aqueous environment.

A pH modifier or buffering agent has several advantages, including maintenance of a pleasant and/or safe pH for consumption. The pH modifier may in addition or alternatively provide a sufficiently acidic environment which in turn may provide an anti-microbial environments. This is beneficial as fewer preservatives are required in the composition.

A pH modifier or buffering agent also can maintain the environment at a consistent pH. This is advantageous as particular pH conditions may alter the functional groups, cause derivatisation or chemical reactions of the derivative or extract of cannabis. If the constituent, derivative or extract of cannabis is maintained at the same pH conditions, this reduces changes to the constituent, derivative or extract of cannabis. A particular example of this advantage is that CBD may decompose into THC in acid environments. As THC is a restricted compound in some jurisdictions, it is of advantage to the invention to maintain the pH of the environment to inhibit or reduce this change.

### Disintegration Enhancers

In some embodiments, the water soluble matrix may comprise one or more additives that enhance disintegration and thereby improve the release and bioavailability of the constituent, derivative or extract of cannabis. Such additives may be materials that instantaneously dissolve on contact with an aqueous environment, providing rapid disintegration of the matrix and enhancing the dissolution and bioavailability of the constituent, derivative or extract of cannabis.

In some cases, the disintegration additive may aid the rapid disintegration of the matrix due to the rapid uptake of water from the medium, swelling, and burst effect.

Examples of suitable disintegrants include: croscarmellose, sodium starch glycolate, and crospovidone, povidone (PVP); and the like.

Effervescent agents, such as sodium bicarbonate in conjunction with an organic acid such as citric or tartaric acid may also act to enhance disintegration of the matrix. Contact with an aqueous medium causes effervescence which affects the structure of the matrix, assisting disintegration and release of the constituent, derivative or extract of cannabis.

Disintegration additives such as croscarmellose can be included in the composition in an amount of from about 0.5% to about 8% by weight, based on the total weight of the composition. Effervescent agents will generally need to be included in a greater amounts. For example, the acid may be included in an amount from about 5% to 20%, with the bicarbonate present in an amount from about 5% to 20% (and dependent on the amount of acid), by weight, based on the total weight of the composition.

### Formats of the Composition

The composition may be in any suitable format, including a liquid, a solid including a solid dispersion, or a colloid including an emulsion or micro-emulsion. The composition may also be incorporated into a product, for example a melt, chew, edible food, beverage, capsule, or any other item intended for oral consumption.

### Emulsion and Micro-Emulsion

In some embodiments, the constituent, derivative or extract of cannabis is provided in an emulsion or a micro-emulsion. The emulsion or micro-emulsion may comprise a non-aqueous solution, the constituent, derivative or extract of cannabis and an amphiphilic material or emulsifier. Micro-emulsions provide the benefit the formation of a micro-emulsion is spontaneous (driven by surface tension, entropy gain, and thermodynamic gain). In addition, micro-emulsions have improved long-term stability and are slow to separate, as this is thermodynamically unfavourable.

### Solid Dosage Form

In some embodiments the composition is provided in a solid form. For example, the form of the composition may be a tablet or other unitary form, or a powder, granules, or particles of any size or shape. The composition may then be combined with the liquid to provide a potable beverage. The provision of the composition in solid form enjoys the advantage of improves stability, as the composition may be stored in an air-tight container, such as a sachet, and only opened prior to consumption. In addition, a solid dosage may be free-flowing, loose powder. This provides the advantage that it is easy to store and transport. The user can add the composition to a liquid such as a drink and so can regulate dosage and the effect of the constituent, derivative or extract of cannabis or composition.

In some embodiments, the user can consume the solid composition directly. In such embodiments, the composition may at least partially dissolve in the saliva of the mouth, and may further be swallowed. This means that at least a portion of the composition may be absorbed by the gastric system, as described herein.

The composition as disclosed herein can be formed into a variety of shapes, including pills, tablets, spheres, strips, films, sheets, coins, cubes, beads, ovoids, obloids, cylinders, bean shaped, sticks, or rods. Cross-sectional shapes of the composition can vary, and example cross-sectional shapes include circles, squares, ovals, rectangles, and the like. Such shapes can be formed in a variety of manners using equipment such as moving belts, nips, extruders, granulation devices, compaction devices, and the like.

It is important to note that larger particles of the same composition will take longer to dissolve than smaller particles, due to the smaller surface area to volume ratio. Therefore, the release of the one or more constituent, derivative or extract of cannabis can be controlled by selecting particles of a particular size or particles of different sizes in appropriate proportions. For example, the larger particles could be around 500 μm while the smaller beads could be between 100 and 50 μm. If the intention is to have a short or instant release, then it may be desirable to keep the d90 to between 125 and 150 μm. Flowability will start to become problematic if the d90 drops to 50 μm and a flowability aid will need to be added to the beads. The size of particles as referred to herein may be measured by sieving.

In some embodiments, the composition is in the form of a particle. This includes when the composition is in a solid dosage form. Each particle may have a maximum dimension. As used herein, the term “maximum dimension” refers to the longest straight line distance from any point on the surface of a particle or on a particle surface, to any other surface point on the same particle or particle surface. The maximum dimension of a particle may be measured using scanning electron microscopy (SEM).

In some embodiments, the maximum dimension of each particle of composition is up to about 800 μm. In some embodiments, the maximum dimension of each particle is up to about 2000 μm. In some embodiments, the maximum dimension of each particle is about 200 μm to about 800 μm.

A population of particles may have a particle size distribution (D90) of at least about 100 μm. In some embodiments, a population of particles has a particle size distribution (D90) of at least 50 μm, of at least 60 μm, of at least 70 μm, of at least 80 μm, of at least 90 μm, of at least 100 μm, of at least 110 μm, of at least 120 μm, of at least 130 μm. In some embodiments, a population of particles has a particle size distribution (D90) of at most 720 μm, of at most 740 μm, of at most 760 μm, of at most 780 μm, of at most 800 μm, of at most 820 μm, of at most 840 μm, of at most 860 μm. Sieve analysis may be used to determine the particle size distribution of the particles.

In some embodiments, the composition is a free flowing powder. If necessary for downstream processing of the composition, such as granulation, mixing, or molding, a flow aid can also be added to the composition in order to enhance flowability of the composition. Exemplary flow aids include microcrystalline cellulose, silica, polyethylene glycol, stearic acid, calcium stearate, magnesium stearate, zinc stearate, sodium stearyl fumarate, canauba wax, and combinations thereof. In some embodiments, the flow aid is sodium stearyl fumarate.

When present, a representative amount of flow aid may make up at least about 0.5 wt % or at least about 1 wt %, of the total dry weight of the composition. Preferably, the amount of flow aid within the composition will not exceed about 5 wt %, and frequently will not exceed about 3 wt %, of the total dry weight of the composition.

### Solubilised Formats

Alternatively, in some embodiments, a product is provided in which the composition comprises a liquid component in order to provide a “pre-mixed” or “pre-made drink”. This enjoys the advantage of being more convenient for the user to consume.

In such embodiments, the liquid composition may be in the form of a product, for example a beverage, or an ingredient in a product, for example an edible item.

The composition may be solubilised in any exemplary method described herein.

### Methods of Manufacture

### Spray Drying

Compositions as described herein or components thereof may be prepared by spray drying.

For example, spray dried emulsions comprising the constituent, derivative or extract of cannabis, lipid or solvent, and a coating material can form stable particles, which may be more soluble. The process may also permit high concentrations of the constituent, derivative or extract of cannabis.

Suitable coating materials for spray drying include inert binder/base material/carrier materials, such as maltodextrin, PVA, and gums. These are preferably water soluble materials to form a water soluble particle. In some embodiments, the coating material comprises maltodextrin. The maltodextrin dissolves in an aqueous environment particularly quickly than a coating material that is not water soluble, such as microcrystalline cellulose (MCC).

The emulsion may be prepared and spray dried to form a dry powder using conventional spray drying methods and apparatus.

It is possible to prepare spray dried particles with different compositions to release the constituent, derivative or extract of cannabis at different rates. This may also be achieved by providing particles of different sizes.

Advantageously, spray drying is a cheap manufacturing process, and provides particles which are easy to handle.

### Extrusion

In some embodiments, the composition or component thereof is formed by extrusion, such as hot melt extrusion.

For this process, the first step is to combine the components into hopper after having pre-mixed the solid components together. This would include, for example, a constituent, derivative or extract of cannabis, lipid or solvent, optionally a water soluble matrix material, plasticizer (e.g. glycerine) and/or flavour. In some embodiments, the size of the particles of the solid ingredients prior to extrusion is no greater than about 1000 μm, or no greater than about 800 μm, to ensure that the particles do not take unduly long to melt when inside the extruder.

In some embodiments, the composition to be extruded comprises a dry polymer, selected from cellulosic/starch-based polymers/PVP/HPC, and an active component, such as CBD.

The application of heat and moulding by the extrusion process causes the constituent, derivative or extract of cannabis to be converted into an amorphous form. The extrusion process “breaks” the crystalline structure of CBD.

In some embodiments, the ratio of water soluble matrix material to plasticizer, if a plasticizer is included, should be around 80:20. Also, if used, a plasticizer should be selected that is compatible with the water soluble matrix material used.

When exiting the hot melt extruder, the mix will harden and form a spaghetti-like string that will need further milling and optionally spheronisation to produce particles that will flow freely. The optimal particle size distribution for good flowability has an approximate d90 of 200 μm.

All percentages by weight described herein (denoted wt %) are calculated on a dry weight basis (DWB), unless explicitly stated otherwise. All weight ratios are also calculated on a dry weight basis. A weight quoted on a dry weight basis refers to the whole of the extract or slurry or material, other than the water or other solvent, and may include components which by themselves are liquid at room temperature and pressure, such as glycerol. Conversely, a weight percentage quoted on a wet weight basis (WWB) refers to all components, including water or other solvent.

For the avoidance of doubt, where in this specification the term “comprises” is used in defining the invention or features of the invention, embodiments are also disclosed in which the invention or feature can be defined using the terms “consists essentially of” or “consists of” in place of “comprises”. Reference to a material “comprising” certain features means that those features are included in, contained in, or held within the material.

The above embodiments are to be understood as illustrative examples of the invention. It is to be understood that any feature described in relation to any one embodiment may be used alone, or in combination with other features described, and may also be used in combination with one or more features of any other of the embodiments, or any combination of any other of the embodiments.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A composition comprising one or more constituent, derivative or extract of cannabis, a lipid or a solvent in which the one or more constituent, derivative or extract of cannabis is at least partially soluble, and an additive which slows or inhibits crystallisation of the one or more constituent, derivative or extract of cannabis.

2. A composition as claimed in claim 1, comprising a liquid in which the one or more constituent, derivative or extract of cannabis is at least partially dissolved.

3. A composition as claimed in claim 1, wherein the lipid is one or more selected from the group consisting of: oils, long or short chain fatty acids, which may be saturated or unsaturated, and long or short chain triglycerides.

4. A composition as claimed in claim 1 or claim 2, wherein the solvent comprises an amphiphilic component such as an alcohol.

5. A composition as claimed in any one of claims 1 to 4, wherein the one or more constituent, derivative or extract of cannabis is encapsulated.

6. A composition as claimed in claim 5, wherein the encapsulation is by a molecular encapsulant, such as a cyclodextrin.

7. A composition as claimed in claim 5, wherein the encapsulation is by a micelle comprising a surfactant.

8. A composition as claimed in claim 7, wherein the surfactant is selected from the group consisting of long chain triglycerides (such as C16-C18 triglycerides), linoleic acid, glyceryl monooleate; and sodium lauryl sulfate (sodium dodecyl sulfate, SLS, or SDS), docusate sodium, lecithin, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®) and sugar esters.

9. A composition as claimed in claim 7 or claim 8, wherein the composition comprises surfactant in an amount of from about 0.5 to about 10% by weight, based on the total weight of the composition.

10. A composition as claimed in any one of claims 1 to 9, wherein the additive is selected from the group consisting of polyvinylpyrrolidone (PVP), hydroxypropyl cellulose (HPC), methyl cellulose (MC), hydroxypropyl methyl cellulose (HPMC), poloxamer (F68), polyvidon, Hydroxypropyl methylcellulose acetate succinate (HPMC-AS), or a combination thereof.

11. A composition as claimed in any one of claims 1 to 10, wherein the one or more constituent, derivative or extract of cannabis is selected from the group consisting of: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

12. A composition as claimed in any one of claims 1 to 11, wherein the constituent, derivative or extract of cannabis is present in an amount of from about 0.1 to about 30% by weight, based on the total weight of the composition.

13. A composition as claimed in any one of claims 1 to 12, having enhanced bioavailability properties in the gastric system.

14. A composition as claimed in any one of claims 1 to 13, comprising one or more components enhancing enterocyte intestinal absorption of the one or more constituent, derivative or extract of cannabis.

15. A composition as claimed in any one of claims 1 to 14, comprising one or more components increasing intestinal lymphatic transport of the one or more constituent, derivative or extract of cannabis.

16. A composition as claimed in claim 15, comprising a terpene, a grapefruit extract, piperine or a black pepper extract.

17. A composition as claimed in any one of claims 1 to 16, comprising a pH modifier.

18. A composition as claimed in any one of claims 1 to 17, comprising a flavour or sensate.

19. A composition as claimed in any one of claims 1 to 18, comprising a further active agent.

20. A composition as claimed in any one of claims 1 to 19, in the form of a solid unit dosage form, a powder or granules.

21. A composition as claimed in any one of claims 1 to 20, wherein the composition is water dispersible.

22. A composition as claimed in claim 21 or claim 22, in the form of particles having a volume mean diameter of from about 50 to about 500 μm.

23. A composition as claimed in any one of claims 1 to 19, wherein the composition is a solution or colloidal dispersion.

24. A method for preparing a composition, the method comprising combining: one or more constituent, derivative or extract of cannabis; a lipid or a solvent in which the one or more constituent, derivative or extract of cannabis is at least partially soluble; and an additive which slows or inhibits crystallisation of the one or more constituent, derivative or extract of cannabis.

25. A method as claimed in claim 24, wherein the combination of the one or more constituent, derivative or extract of cannabis, the lipid or solvent in which the one or more constituent, derivative or extract of cannabis is at least partially soluble, and the additive which slows or inhibits crystallisation of the one or more constituent, derivative or extract of cannabis is dried to form a water soluble or water dispersible solid composition.

26. A method as claimed in claim 25, wherein the combination is spray dried to form the composition.

27. A beverage for providing gastric delivery of one or more constituent, derivative or extract of cannabis, comprising a composition as claimed in any one of claims 1 to 23.

28. Use of a composition as claimed in any one of claims 1 to 23 to form a beverage for providing gastric delivery of one or more constituent, derivative or extract of cannabis by introducing the composition into a liquid.
